(19) United States
(12) Patent Application Publication
(10) Pub. No.: US 2022/0195357 A1
(43) Pub. Date: Jun. 23, 2022

(54) DEVICE AND METHODS FOR FREE FLOATING MACROALGAE CULTIVATION OFFSHORE

(71) Applicant: Ramot at Tel-Aviv University Ltd., Tel Aviv (IL)

(72) Inventors: Alexander GOLBERG, Tel Aviv (IL); Alexander CHEMODANOV, Tel Aviv (IL); Alexander LIBERZON, Tel Aviv (IL)

(21) Appl. No.: 17/310,519

(22) PCT Filed: Feb. 5, 2020
(86) PCT No.: PCT/IL2020/050139

§ 371 Date: Aug. 6, 2021

Related U.S. Application Data
(60) Provisional application No. 62/803,118, filed on Feb. 8, 2019.

(51) Int. Cl. C12M 1/00; A01G 33/00

(52) U.S. Cl. CPC C12M 21/02; C12M 23/26; C12M 23/56; C12M 29/06; A01G 33/00; C12M 23/38

(57) ABSTRACT

The present invention provides novel devices, systems and methods for cultivating macroalgae in a waterbody, more particularly in the sea/offshore.

### FIELD OF INVENTION

The present invention is in the field of marine agriculture. More specifically, the present invention provides devices and methods for cultivation of macroalgae in waterbodies, e.g., in sea/offshore.

### BACKGROUND

Although agriculture is the primary method to produce biomass for food, biochemicals, and biofuels, the European Biorefinery Joint Strategic Research Roadmap for 2020 indicates that “a key issue for biomass production in Europe is land availability”. Accordingly, countries with limited arable land for energy crops cultivation can find marine macroalgae farming a useful alternative that can provide a sustainable feedstock biomass for downstream processing in biorefineries (Jung et al., 2013; Suganya et al., 2016; Goh et al., 2010; Ben Yahmed et al., 2016). For instance, the methodology of macroalgae biorefinery design for rural areas in developing countries has been developed (Golberg et al., 2012 and 2014). Nevertheless, a key challenge in the field of macroalgae biorefinery remains to be the sustainable production of the biomass (Skjermo et al. 2014; Roesijadi et al., 2008; Jiang et al., 2011).

Offshore cultivation of macroalgae is one sustainable strategy to produce bioenergy and bioproducts without using arable land and scarce freshwater resources (Lehahn et al., 2016; Fernand et al., 2016). The concepts of offshore marine biomass cultivation include farms for kelp growth, tidal flat farms, floating seaweed cultivation settings (Bird, 1987a and 1987b), ring cultivation systems (Buck et al., 2004) and most recently wind-farm integrated systems (Buck et al., 2017) and underwater ropes (Camus et al., 2016).

Following the success of on-land photobioreactors in providing high biomass yields when the major cultivation parameters of temperature, light, mixing, and nutrients were controlled (Zijffers et al., 2010; Cuaresma et al., 2011), theoretically possible intensified offshore cultivation methods were proposed (Golberg et al., 2015; Hirayama et al., 2004). However, intensification methods that control key parameters offshore have not been demonstrated in the field.

However, current systems and devices suffer from carious disadvantages and drawbacks, such as: not providing active gas or nutrients exchange offshore; do not use the whole volume of water for cultivation offshore; do not actively remove ephyfits with movements; do not fully use light—since not all plants are evenly exposed.

Accordingly, a need exists for methods, devices and systems for intensified macroalgae cultivation offshore that overcome the problems of known systems and methods.

### SUMMARY OF INVENTION

In a first aspect, the present invention provides an open-water apparatus for growing macroalgae in a body-of-water (waterbody), such as sea/offshore, the apparatus comprising: (a) a growing/cultivation cage/reactor for positioning in the water/waterbody, having permeable walls and bottom enabling free flow of water, gas and nutrients from the waterbody into the growing cage and vise-versa; and (b) a macroalgae suspending and mixing system designed to mix/tumble/suspend water in the cage from bottom to top and consequently the macroalgae grown therein, by streaming gas from bottom of the cage via gas flow outlets; wherein the apparatus is designed for free-floating growing of the macroalgae.

The apparatus of the invention may be modified in any number of ways and may comprise additional add-ons and supplements as defined herein, either integral or not. Such additional add-ons may include, e.g., a power source, and/or a floatation device/mechanism for maintaining the cage floating at water-surface, or at a desired depth at which the upper surface of the water in the cage is still exposed to sunlight. Thus, any system comprising the structural components of the above apparatus, as well as one or more of the other components optionally further comprised within the apparatus, is also to be considered as part of the invention.

Thus, in a second aspect, the present invention provides a system for growing macroalgae in a body-of-water, such as the sea/offshore, the system comprising: (a) a growing/cultivation cage/reactor for positioning in the water/waterbody, having permeable walls and bottom enabling free flow of water, gas and nutrients from the waterbody into the growing cage and vise-versa; (b) a macroalgae suspending and mixing system designed to mix/tumble/suspend water in the cage from bottom to top and consequently the macroalgae grown therein, by streaming gas from bottom of the cage via gas flow outlets; (c) a power source; and (d) a floatation device/mechanism for maintaining the cage floating at the water-surface, or at a desired depth at which the upper surface of the water in the cage is still exposed to sunlight, wherein the system is designed for free-floating growing of the macroalgae.

In a third aspect, the present invention relates to a method for growing macroalgae in a body-of-water, such as the sea/offshore, the method comprising the steps of: (i) positioning in the body-of-water an apparatus as defined above; (ii) placing an inoculum of macroalgae in the growing/cultivation cage/reservoir cage; (iii) activating the macroalgae suspending and mixing system for tumbling/suspending the water in the cultivation cage from bottom to top to thereby expose in a cyclic manner different portions of the water in the cage, and consequently the macroalgae grown therein, to sunlight, wherein: (1) the amount, intensity and speed of gas streamed into the cultivation cage by the suspending and mixing system is determined according to the type, density, and growing stage of the macroalgae; and (2) the tumbling is conducted continuously until a desired density/amount of the macroalgae in the cultivation cage is achieved; and (iv) harvesting the macroalgae.

### BRIEF DESCRIPTION OF THE DRAWINGS

FIGS. 1A-1C are illustrations of a cultivation reactor according to some embodiments of the invention: FIG. 1A is a schematic design of the reactor with intensification with tumbling, mixing and water exchange; FIG. 1B is an image of an exemplary reactor for intensified cultivation; and FIG. 1C illustrates the external airlifts for water exchange enhancement for the cultivation reactor.

FIGS. 2A-2C are images of actual cultivation reactors with external airlifts used. FIG. 2A shows an exemplary reactor; FIG. 2B shows deployment of reactors with algae at the cultivation site; and FIG. 2C shows solar dried Ulva biomass.

FIGS. 3A-3B are illustrations of cages for extensive cultivation. FIG. 3A are images of a cage (upper and lower angels); and FIG. 3B is an illustration of a cultivation bottle/container for on-land cultivation under controlled conditions, schematically showing trajectories for measured Thalli velocity.

FIGS. 4A-4B are graphs illustrating illumination and temperature (FIG. 4A) or profile inside the cultivation reactor (FIG. 4B). Information was recorded with 15 min resolution continuously.

FIG. 5 is a graph showing temperature profile in aerated and non-aerated cultivation cages (June-July 2017).

FIGS. 6A-6D are graphs illustrating daily growth rates of algae in a cultivation reactor according to the invention: FIG. 6A illustrates Ulva growth in intensified and extensive cultivation systems in the sea (n=3); FIG. 6C illustrates growth productivity; and FIGS. 6B and 6D illustrate the minimum, maximum and average measurements.

FIG. 7 is a graph showing DGR of Ulva biomass in a controlled, inland 1.5 L cultivation bottles with and without tumbling with air and mixing. Seawater and excess of nutrients was changed daily.

FIGS. 8A-8B are graphs showing monosaccharides content in acid hydrolysate of a biomass harvested from two cultivation cages—one with intensified cultivation and the other with extensive cultivation (May 2017): FIG. 8A shows the concentration of the monosaccharides; and FIG. 8B shows p-values for comparison (Student-t) of the content between the two cultivation methods. n=6 for intensified cultivation and n=10 for extensive cultivation. The monosaccharides are: Gal-galactose, Glu-glucose, GluA-glucuronic acid, SA-other sugar acids, Rha-rhamnose, Xyl-xylose, Fru-fructose, and UA-uronic acid.

FIGS. 9A-9C are graphs showing monosaccharides content comparison between biomass harvested from cultivation cages with intensified cultivation or extensive cultivation (May 2017). The monosaccharides are: Gal-galactose, Glu-glucose, GluA-glucuronic acid, SA-other sugar acids, Rha-rhamnose, Xyl-xylose, Fru-fructose, UA-uronic acid.

### DETAILED DESCRIPTION

The growing of human population forces to find new and improved ways to grow food and produce energy. However, arable land is scarce and decreases. One solution is offshore growing of macroalgae, which has its own problems and difficulties. The present invention provides new device, system and method for efficient and cost effective offshore macroalgae cultivation.

It has known that cultivation in standing water leads to reduced growth and productivity, and fast decay of the grown plant. Accordingly, in order to increase growth and yield, and prevent undesired death of the grown plant, the water in the cultivation tank needs to be constantly mixed and preferably replaced from time to time. Tumbling of the water, e.g., with air, causes motion of the water and the algae floating therein in the cultivation reactor thereby further reducing shading limitations, and increasing exposure to light and available dissolved nutrients, thus enhancing photosynthesis and productivity. Tumbling with air may also prevent development of competitive macroalgal grazers and epiphytes, such as diatoms.

It should be noted that although the present description and examples below explicitly demonstrate growth of the Ulva sp. macroalgae, the device and method of the present invention are suitable for growing any type of algae and macroalgae.

Notably, in nature Ulva grows primarily attached to hard substrates. However, it is also found growing in a floating stage within the water column. Accordingly, cultivation of free-floating algal biomass enables to use water volumes for cultivation instead of large growing areas, thereby reducing the amount of areas required for cultivation. The term “cultivation” as used herein refers to all types of water agriculture for growing macroalgae, including offshore cultivation.

In a first aspect, the present invention thus provides an open-water apparatus for growing macroalgae in a body-of-water (waterbody), preferably in the sea/offshore, the apparatus comprising: (a) a growing/cultivation cage/reactor for positioning in water, having permeable walls and bottom enabling free flow of water, gas and nutrients from the waterbody into the growing cage and vise-versa; and (b) a macroalgae suspending and mixing system designed to mix/tumble/suspend water in the cage from bottom to top and consequently the macroalgae grown therein, by streaming gas from bottom of the cage via gas flow outlets; wherein the apparatus is designed for free-floating growing of the macroalgae.

In certain embodiments, the open-water apparatus disclosed herein comprises: (a) a growing cage for positioning in the water, having permeable walls and bottom enabling free flow of water, gases and nutrients from the water surrounding the cage, including any waste streams or other streams that flow into the body-of-water near the cage, into the growing cage and vise-versa; (b) a suspending system designed to tumble/suspend the water in the cage from bottom to top and consequently the macroalgae grown therein, by streaming gas, such as air, oxygen etc., from the bottom of the cage via gas flow outlets (i.e. an internal airlift). Such a suspension system enables to: (1) expose in a cyclic manner different portions of the water in the growing cage, and consequently the macroalgae grown therein, to sunlight; (2) optionally aerate the water within the cage; and (3) generate water flow from the waterbody into the growing cage and vise-versa; and (c) optionally, a floatation device/mechanism for maintaining the growing cage floating at water-surface, or at any desired depth at which the upper surface of the water in the cage is still exposed to sunlight, wherein the apparatus is designed for free-floating growing of the macroalgae.

In preferred embodiments, the apparatus of the invention is aimed at growing/cultivating macroalgae in the open sea or offshore, e.g., in order to reduce growing costs and complexity by using real seawater and natural nutrients and aeration provided by the open sea. Yet, the apparatus can be used for growing/cultivating macroalgae in any marine environments, including natural and artificial lakes and rivers, artificial tanks, and pools.

The term “free flowing water” as used herein should be understood according to the designated growing location, and thus may refer to seawater, sweet water, wastewater, industrial water, or used-water, e.g. from plants or oil platforms, etc.

The term “free-floating” as used herein means that the macroalgae grows within the growing cage in a floating state, i.e., not anchored to a stationary, non-moving, base. Yet, this term also includes the possibility wherein the macroalgae is grown in the cage while being attached to a floating substrate, such as capsules, e.g., gelatin capsules or plastic bubbles. Such a configuration enables growing the macroalgae in a floating state while providing the macroalgae a platform to cling to.

The terms “water-surface” and “upper water-surface” as used herein interchangeably, refer to the upper surface of the water of the body-of-water, and is intended to describe that the cage of the apparatus of the invention is located/floats essentially at the uppermost water-level. In specific embodiments, when the body-of-water is the sea or ocean, the water-surface is sea-level.

The growing cage of the apparatus of the invention, according to any one of the embodiments above, can be in any desired shape, e.g., in a boxed-shaped, U-shaped, cylindered-shaped, etc., and may be in any size, according to the need, type of macroalgae, and comfort of the growing farmer. For instance, the volume of the growing cage may vary from 1 to 10000, 50000, 100000, 250000, 500000, or 1000000 liters. It should be noted that the bigger the growing cage is, the more gas outlets are needed in order to maintain efficient turbulence/suspension of the growing macroalgae within the cage. Similarly, if used, more water-pumps, external airlifts and bigger heating/cooling unit(s), are needed.

It should further be noted that due to the many possible configurations and shapes of the growing cage, the boundaries between the “walls” and the “bottom” of the cultivation cage may be vague or even none, i.e., the bottom and walls of the cage may constitute a single unit. Also, when the cage has a cylindered shape, there might not be any distinguish between the “walls”, “bottom” and “top” of the cage.

Light is an essential component in growing of macroalgae. As such, it is essential to make sure that all the macroalgae in the growing cage are exposed to a sufficient amount of light, e.g., sunlight, and for a sufficient time period daily. Since water do not necessarily block light, it may be beneficiary to use a growing cage with transparent/light-permeable walls so that light may penetrate through and illuminate the macroalgae not only from the top surface of the cage. Accordingly, in certain embodiments of the apparatus of any of the embodiments above, the permeable walls of the growing cage are transparent/light-permeable to enable penetration of light into the cage. The terms “transparent” and “light-permeable” as used herein interchangeably refer to the ability of an object to pass light therethrough, at any wavelength or any desired wavelength according to need. For instance, the transparent walls may be designed to enable passage of only UV light or wavelengths of between about 400 nm to about 700 nm, which are optimal for photosynthesis.

The floatation device/mechanism can be used to place the cultivation cage at any desired depth according to need, dependent on various conditions, such as lighting conditions and the amount of light reaching the macroalgae inside the cage, water temperature, sea condition—when the sea is wavy, it might be advisable to lower the depth of the cage to prevent possible damage, the viscosity and clarity of the water (when the water is clearer, the cage can be placed dipper while still receiving sufficient amount of sunlight), amount, type and growing stage of the macroalgae, etc.

In certain embodiments of the apparatus of the invention, the permeable walls are non-selective permeable walls. Alternatively, the permeable walls are selective, i.e., designed to limit the passage of specific salts, nutrients, or any other substance that is considered harmful or damaging to the macroalgae.

As explained above, it is desirable to mix the water within the cultivation cage in order to constantly expose the grown macroalgae to light, aerate the water, and homogenize nutrients within the cage. This is achieved in part by the free flow of water in and out of the cage. However, in order to make this constant and to obtain equal mixing, a macroalgae suspending and mixing system is used. Nevertheless, under certain conditions, e.g., when the amount of macroalgae in the cage rises above a certain level, nutrients' level decreases, or the temperature within the cage rises to a certain point, there might be a need to increase the mixing. This can be done by any suitable means, such as water-pump and/or airlift(s). In addition, in certain scenarios, e.g., due to macroalgae high density in the case, poor content of nutrients in the surrounding water, or poor water flow, the clearance of waste from the growing cage, and introduction of nutrients and gases, such as oxygen and CO₂, into the growing cage, by the free flow of water through the walls of the cage may be insufficient. As such, it might be necessary to step in and actively replace the water in the growing cage, e.g., by introducing fresh water from the surrounding into the cage, e.g., by pumping. In certain embodiments, the apparatus of any of the embodiments above thus further comprises a water-pump for exchanging the water in the growing cage with fresh water, e.g., from outside the cage, optionally from a remote location, e.g., a few meters from the cage.

As stated above, the walls of the cage are water permeable and allow free flow of water from the outside to the inside of the cage and vise-versa. However, as noted above, under certain conditions such water flow might not suffice so that there will be a need to increase water circulation/replacement, e.g., due to large amount of macroalgae in the cage which require more nutrients and waste removal and/or temperature adjustment, water content that raises a need for faster water exchange in the cage, etc.

Accordingly, in certain embodiments, the apparatus of any of the embodiments above further comprise at least one external airlift for water exchange with fresh water from outside the cage—and optionally turbulence enhancement in the growing cage. Such external airlift(s) may comprise gas pump and gas flow outlets that are located externally to the growing cage, e.g., at its bottom. The external airlift(s) enables to improve various conditions, such as (a) nutrient enrichment of the water in the cage; (b) waste removal from the cage; and (c) optionally, tumbling of the water in the cage.

As noted above, under certain conditions there might be a need to increase water circulation inside the cage. An airlift is one option of doing so. One of the advantages of an airlift is that it can be designed to bring water from lower levels of the waterbody, thereby delivering colder and richer water from below, which both assists in temperature control and nutrient levels inside the cage. Notably, the flow of water from the airlift can further assist in the tumbling and mixing of the macroalgae inside the cage and can have a synergistic effect when combined with the air-based macroalgae suspending and mixing system.

The apparatus of the invention as described herein may be modified in various ways in order to improve its operation, the growing of the macroalgae, biomass production, and the content of the grown biomass. For instance, the apparatus may further comprise an artificial light source for emitting light onto the macroalgae in case there is insufficient light from the sun, or for emitting ultraviolet light for sterilization. It may also be used to emit light at desired wavelengths to improve macroalgae's growth and/or eliminate bacteria or viruses.

In certain embodiments, the apparatus of the invention further comprises an artificial light source. As noted, the purpose of the artificial light source is to provide an alternative or supplementary source of light in addition to sunlight to maximize macroalgae growth/cultivation and reduce dependency on the weather (clouds, rain, etc.). Another usage of such artificial light source is to provide a specific wavelength(s) for specific purposes, such as for enhancing production of certain proteins or other products within the grown macroalgae, or for sanitation, i.e., killing viruses or bacteria, or inhibiting their growth. In specific embodiments, the artificial light source is placed inside the cage to allow light emission onto macroalgae grown therein. In alternative or supplementary embodiments, the artificial light source is placed externally to the cage. It may be placed at the bottom, side walls and/or the top of the cage, or any combination thereof.

Growing of the macroalgae in the growing cage may cause the total weight of the cage to increase over time. This means that the cage, which might float at the beginning of the growing process at a certain depth, might sink as the macroalgae grows and its mass increases. Accordingly, the floatation state of the growing cage may need to be adjusted during time. This may be done by adding floatation units (e.g., buoys) to the cultivation cage or by inflating floatation balloons connected to the cage. Alternatively, the cage may be connected to a fixed frame maintaining the cage level/depth constant. A reversed problem may arise when harvesting the macroalgae or when diluting it.

Accordingly, in certain embodiments, the apparatus of any one of the embodiments herein comprises a floatation device/mechanism that is designed to maintain the growing cage at water-surface level, or at a desired depth at which the upper surface of the water in the cage is still exposed to sunlight. It should be noted that the floatation device may comprise inflatable containers that may be filled or depleted with gas in order to adjust the floating level/depth of the cage. Alternatively, it may comprise tanks that can be filled or depleted with water (as in a submarine) in order to control the floating level/depth of the cage. Notably, the control of the flotation of the cage may be manual and/or automatic according to need and/or according to data received from various integral or external sensors associated with the apparatus of the invention. The floatation device/mechanism may be an integral part of the cage or an add-on, or an entirely separate unit that the cage is to be attached to.

In certain embodiments, the apparatus according to any of the embodiments herein further comprises a light sensor designed to measure the amount of light within the cage and (i) adjust the floating level/depth of the cage accordingly using the floatation device/mechanism; and/or (ii) activate an artificial light source when present. It should be noted that such a floatation mechanism may be an integral part of the apparatus, e.g., constitutes the framing and/or part of the walls, bottom or top of the cage. Notably, when the apparatus does not include such a floatation mechanism, it may be connected to such a mechanism or to an external frame/rope/weights designed to maintain the cage at a desired location and depth.

Another modification of the apparatus of any of the embodiments herein is the use of a heating/cooling unit associated with a thermostat measuring the temperature within the growing cage. Such a heating/cooling unit can assist in controlling the water temperature within the growing cage and adjusting it to optimal or near optimal growing temperature. In certain embodiments, the heating/cooling unit uses external water for heating or cooling the water inside the growing cage, e.g., by pumping surrounding water into the growing cage to modify, i.e., lower or increase, the temperature of the water currently inside the cage. Such external water can be pumped from a remote location relative to the growing cage, e.g., from a few meters below the cage (which are usually colder water) or from a nearby stream, which may have colder or warmer water, depending on the type of stream. Accordingly, in certain embodiments, the apparatus of any one of the embodiments herein further comprises a heating/cooling unit associated with a thermostat measuring the water temperature within the growing cage.

As explained above, water temperature adjustment within the cultivation cage/reactor assists to facilitate optimal growth conditions of the macroalgae and/or of specific components therein. It may also assist in reducing infection, e.g., by maintaining a temperature that inhibits bacteria/viruses' growth and proliferation. Notably, the water temperature within the cage is dependent on the surrounding water's temperature, water flow, sunlight, amount of macroalgae inside the cage, and operation of any airlifts or water-pumps, etc. Nevertheless, the presence of a heating/cooling unit can further aid in controlling the water temperature within the cultivation cage/reactor. In specific embodiments, the artificial light source may also serve as a heating unit.

Still another possible modification is the use of an internal power source/supply for powering the different components of the apparatus, such as different pumps, the suspending and mixing system, artificial light source, and heating/cooling unit. Such a power source/supply may be rechargeable batteries and/or solar panels. Accordingly, in certain embodiments, the apparatus of any one of the embodiments herein further comprises a power source. The power source may be a rechargeable power source, such as solar- or aquatic-based, to enable the apparatus to be autonomous without the need to provide external power and/or replace batteries.

In certain embodiments, it is desirable to keep the cultivation cage closed in order to prevent unintentional escape of macroalgae from the cage, e.g., due to waves or streams, and/or to prevent unintentional penetration of, e.g., fish and birds or other contaminants into the cage, which may damage the macroalgae and its' growth. As such, the growing cage may be equipped with a lid or cover that may be removed/opened when needed. Accordingly, in certain embodiments of the apparatus of any of the embodiments herein, the growing cage is a closed cage further having a non-selective permeable and transparent (light-permeable) cover/lid.

The growing cage may be a closed cage with a permanent top/“roof” section. This top section may be non-selective permeable and transparent. In certain embodiments, the cage comprises an opening in one (or more) of the walls or bottom (or through the openable top), through which macroalgae is inserted into the cage for growing, and eventually removed when needed, e.g., for harvesting.

In specific embodiments, the permeable walls, bottom, and optional cover of the growing cage are made of a (dense) mesh designed to prevent macroalgae from exiting the cage, and fish from entering the cage and/or grazing on the macroalgae through the mesh. In certain embodiments, the permeable walls, bottom, and optional cover of the growing cage are non-selective permeable walls.

The apparatus of the invention can be used to grow macroalgae at any aqueous environment, such as the open sea, artificial seawater pools/reservoirs, wastewater pools/reservoirs, freshwater pools, rivers, industrial waters, etc. Accordingly, the permeable walls of the cage allow passage and free flowing of any water type, such as seawater, sweet water, wastewater, or used-water, etc. In specific embodiments, the permeable walls of the cultivation cage further enable penetration of light into the cage.

The terms “body-of-water” and “waterbody” as used herein interchangeably refer to any significant accumulation of water, such as large natural ones like oceans, seas and lakes, and smaller ones like pools of water and wetlands. A body-of-water does not have to be still or contained, and may include also rivers, streams, canals, and other geographical features where water moves from one place to another. Body-of-water also includes man-made water accumulation, such as artificial pools of seawater, freshwater, wastewater, and industrial water reservoirs, and also artificial lakes, rivers and streams.

As noted, the apparatus of the invention comprises an integral suspending and mixing system designed to mix the water within the cage and subsequently the macroalgae grown therein, thereby enabling exposure of all macroalgae to (sun)light and to maintain homogeneity of nutrients within the cultivation cage. This suspending and mixing system is based on streaming gas (such as air, oxygen etc.) from, e.g., the bottom of the cage via gas flow outlets at the bottom of the growing cage. The actual mechanism for streaming the gas may be either an external mechanism, not part of the apparatus of the invention or may be an integral part thereof. Accordingly, in certain embodiments of the apparatus of any of the embodiments herein, the suspending and mixing system comprises a gas-blowing mechanism and gas pipes positioned essentially at the bottom of the cage.

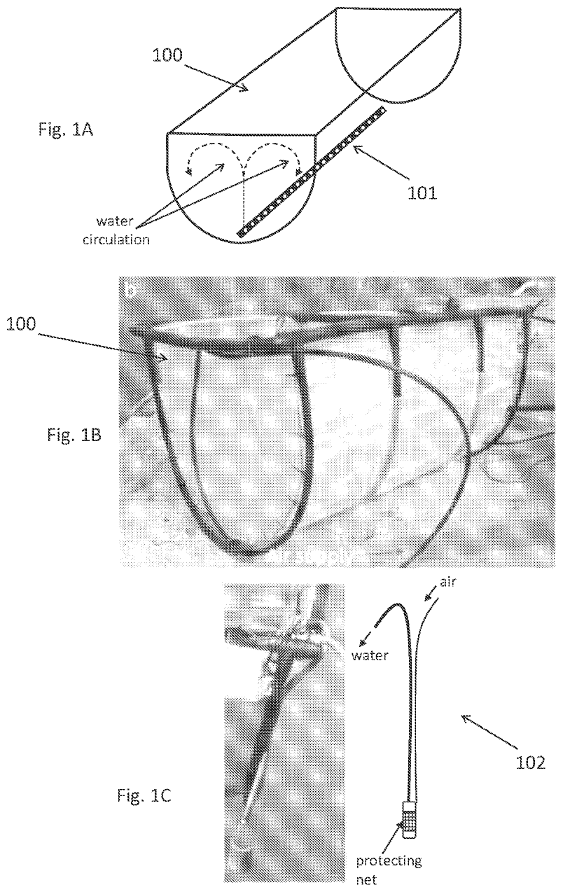

The term “gas” or “air as used herein interchangeably refer to any gaseous matter, such as air, oxygen, nitrogen etc. Non-limiting examples of a gas-blowing mechanism include an air pump, a compressor, or even a compressed gas container that discharges the gas. It should be noted that the positioning of the gas pipes is determined according to the shape and size of the cage to enable optimum tumbling and mixing of the water and microalgae inside the cage. For instance, the apparatus may comprise a single pipe positioned in the middle at the bottom of the cage (see illustrated in FIG. 1A). Alternatively, two parallel pipes may be evenly spaced apart at the bottom of the cage. The pipes may be positioned directly at the bottom of the cage or elevated therefrom in order to improve the mixing. Each gas pipe may have a single raw of air-holes or two parallel air holes, creating two air streams that facilitate even mixing. In specific embodiments, the framing of the cage constitutes the air pipes.

All modifications and additions to the apparatus of any of the embodiments above may be designed as add-ons that are attached/mounted/connected/associated with the apparatus or designed as an integral part thereof.

The rate of gas streaming and the amount of gas streamed into the growing cage can be controlled, manually and/or automatically according to various parameters, such as light intensity, water turbidity, amount/density of the macroalgae in the growing cage, nutrients and gases content in the water in the cage, the macroalgae growing stage, etc. For instance, as the density of macroalgae increases and/or the light intensity decreases, so the amount of gas being streamed is increased in order to increase turbulence of water in the cage. Such parameters may be measured using integral or external sensors associated with the apparatus of the invention, and may be controlled by, e.g., a computing system, which may be an integral part of the apparatus. Accordingly, in certain embodiments, the apparatus of any of the embodiments herein further includes a computing system comprising a memory and a processor. Such a computing system, when present is designed to control any one of the various systems and units within the apparatus and/or associated therewith, such as: the floatation mechanism; artificial light source(s); water-pump(s); external airlift(s); and/or heating/cooling unit, based on data received from, e.g., light sensor(s), temperature sensor(s) and thermostat(s), weight sensor(s), or any other measuring device/sensor

In certain embodiments, the apparatus of any of the embodiments above further comprises, or is associated with, a computing system comprising a memory and a processor designed to control any one of: the macroalgae suspending and mixing system; a floatation mechanism; an artificial light source(s); a water-pump(s); an external airlift(s); and/or heating/cooling unit(s). The computing system may be an integral part of the apparatus or may be associated therewith, either directly via a cable or remotely via wireless communication. In certain embodiments, some components of the computing system are integrated within the apparatus while other components are located remotely and are associated, wirely or wirelessly, to the components within the apparatus.

In specific embodiments, the present invention provides an open-water apparatus for growing macroalgae in the sea/offshore, the apparatus comprising: (a) a growing/cultivation cage/reactor for positioning in seawater, having permeable walls and bottom enabling free flow of seawater, gas and nutrients from the sea into the growing cage and vise-versa; (b) a macroalgae suspending and mixing system designed to mix/tumble/suspend seawater in the cage from bottom to top and consequently the macroalgae grown therein, by streaming gas from bottom of the cage via gas flow outlets; (c) a floatation device/mechanism for maintaining the cage floating at seawater-surface, or at a desired depth at which the upper surface of the seawater in the cage is still exposed to sunlight; (d) at least one external airlift for seawater exchange, and optionally turbulence enhancement, in the growing cage; and (e) a water-pump for seawater exchange in the growing cage; wherein the apparatus is designed for free-floating growing of the macroalgae.

In further specific embodiments, the above apparatus further comprises at least one of (a) an integral artificial light source and optionally a light sensor designed to measure the amount of light within the cage and: (i) adjust the floating level/depth of the cage accordingly using the floatation device/mechanism; and/or (ii) activate an artificial light source when present; (b) a power source; (c) a heating/cooling unit associated with a thermostat measuring the water temperature within the growing cage; and (d) a computing system comprising a memory and a processor designed to control any one of: a floatation mechanism; an artificial light source(s); a water-pump(s); an external airlift(s); and/or heating/cooling unit(s).

The present invention further provides an open-water system for growing macroalgae in a body-of-water, such as the sea/offshore, the system comprising: (a) an apparatus according to any of the embodiments herein comprising: a growing/cultivation cage/reactor for positioning in water, having permeable walls and bottom enabling free flow of water, gas and nutrients from the waterbody into the growing cage and vise-versa; and a macroalgae suspending and mixing system designed to mix/tumble/suspend water in the cage from bottom to top and consequently the macroalgae grown therein, by streaming gas from bottom of the cage via gas flow outlets; (b) a power source; and (c) a floatation device/mechanism for maintaining the cage floating at upper water-surface, or at a desired depth at which the upper surface of the water in the cage is still exposed to sunlight, wherein the system is designed for free-floating growing of the macroalgae.

In specific embodiments, the above system further comprises at least one of an integral artificial light source; and a light sensor designed to measure the amount of light within the cage and (i) adjust the floating level of the cage accordingly using the floatation device/mechanism; and/or (ii) activate the artificial light source when present.

In further specific embodiments, the above systems further comprise at least one of (a) a water-pump for water exchange in the growing cage (with fresh water from outside the cage); and (b) at least one external airlift for water exchange (with fresh water from outside the cage), and optionally turbulence enhancement, in the growing cage.

In yet further specific embodiments, the above systems further comprise a heating/cooling unit associated with a thermostat measuring the water temperature within the growing cage.

In specific embodiments, the system according to any of the embodiments above further comprises a computing system comprising a memory and a processor designed to control any one of: the floatation mechanism; light source; water-pump; external airlift(s); and/or heating/cooling unit based on data received from, e.g., light sensor(s), temperature sensor(s) and thermostat(s), weight sensor(s), or any other measuring device/sensor.

The present invention further provides a method for growing macroalgae in a body-of-water (waterbody), such as a sea/offshore, using the apparatus or system of any of the embodiments above, the method comprising the steps of: (i) positioning in the waterbody a cultivation apparatus according to any one of the embodiments above; (ii) placing an inoculum of macroalgae in the growing/cultivation cage/reservoir cage; (iii) activating the macroalgae suspending and mixing system for tumbling/suspending the water in the cultivation cage from bottom to top to thereby expose in a cyclic manner different portions of the water in the cage, and consequently the macroalgae grown therein, to sunlight, wherein: (1) the amount, intensity and speed of gas streamed into the cultivation cage by the suspending and mixing system is determined according to, e.g., the type, density, and growing stage of the macroalgae; the viscosity and clarity of the water: and the amount of nutrients and oxygen in the water within the cage; or any combination thereof, and (2) the tumbling is conducted continuously until a desired density/amount of the macroalgae in the cultivation cage is achieved; and (iv) optionally, harvesting the macroalgae.

It should be noted that the final step of harvesting the macroalgae is carried out according to need. The harvesting may be of the entire biomass within the growing cage, or part thereof leaving behind enough macroalgae to continue to grow and generate more biomass.

In specific embodiments of the above method, the cultivation apparatus further comprises a water-pump and/or at least one external airlift, and the method further comprises a step of activating the water-pump and/or the at least one external airlift for water exchange enhancement in the cultivation cage, and consequently enriching nutrient level therein, and optionally waste removing therefrom. Such external airlift may also assist in the tumbling of the water in the cage.

In further specific embodiments, the above methods further comprise a step of actively exchanging the water, nutrients and/or waste in the growing cage (according to need) by pumping water into the growing cage, optionally from a remote location in the waterbody, e.g., a few meters from the cage. Such water exchange may increase gases and nutrients concentrations in the cage and reduce waste concentration therein. It may also aid in temperature control of the water in the cage.

In further specific embodiments, the above methods further comprise a step of: (i) adjusting the water temperature within the cultivation cage, e.g., by pumping water into the cage from a remote/deep location in the waterbody and/or by activating a heating/cooling system; and/or adjusting the amount of light reaching the cultivation cage, e.g., by activating an artificial light source associated with the cage, controlling the height/depth of the cultivation cage, and/or controlling the speed of the mixing of the water and macroalgae within the cage.

### Examples

In preliminary experiments to prove the feasibility of the invention, Applicant has used a ˜2 m³ cage deployed in a shallow area in Tel Aviv, Israel, with tumbling and mixing of biomass with air and water supplied by an airlift pump from a deeper water layer. The selected macroalgae was green seaweed Ulva sp. since: (i) it is common on Israel shores; (ii) it showed high biomass productivity; (iii) it is known to produce proteins and starch; and (iv) several Ulva species have demonstrated biomass-fermentation to acetone, ethanol, butanol, and polyhydroxyalkanoates.

### Materials and Methods

### Cultivation Site

Ulva sp. cultivation site was located in a shallow coastal area at the proximity of an electric power plant in Tel Aviv (32°07′00″ N 34°49′00″ E), Israel. This location allowed continuous monitoring of the biomass cultivation site conditions.

### Macroalgae Biomass Inoculum

The model seaweed used in this study belongs to the genus Ulva sp., a green marine macroalgae of worldwide distribution found in the intertidal and shallow waters within the Israeli Mediterranean Sea shores. The exact taxonomic status of the Ulva sp. used in this study suggests a mix of two morphological and genetically similar types, Ulva rigida and Ulva fasciata (Krupnik et al., 2018). Specimens were taken from stocks cultivated in an outdoor seaweed collection at Israel Oceanographic & Limnological Research, Haifa, Israel (IOLR), in 40 L fiberglass tanks supplied with running seawater, tumbling with air, and weekly additions of 1 mM NH₄Cl and 0.1 mM NaH₂PO₄. With each nutrient application, the water exchange was stopped for 24 h to allow for nutrients uptake.

Offshore Cultivation in Cages with Tumbling, Air Mixing and Water Exchange

To test the potential of tumbling, air mixing and external water exchange on the intensification of the Ulva sp. biomass growth, a cultivation system comprising a floating cage 100 equipped with air flow outlets 101 at its bottom for constant aeration was designed (FIG. 1A). The experimental U-shape carcass 100 (working volume 1.785 m³, total illuminated area 2 m², FIG. 1B) was built from high-density polyethylene pipes (Ø=50 and 35 mm) and a Ginigar anti-insect net (25 mesh, FIG. 1B) that effectively prevented fish grazing. Air was supplied to the bottom of the cage through a polyethylene pipe (Ø=20 mm) at 40-45 LPM/reactor or 20-22.5 LPM m⁻³ of water, depending on the load density of the biomass (ranged from 1 kg m⁻³ at the beginning of the cultivation to 4.5 kg m⁻³ at harvesting). Additional water was pumped into the cage from 1 m depth using 4 airlifts 102 made from HDPE Single Wall Corrugated Pipe (Ø=20 mm) and 4/7 PVC pipes. (FIG. 1C). The airlift 102 pumped 11.03 m³ of water per day, which equals to 618.2%/day water exchange in the cage/cultivation reactor.

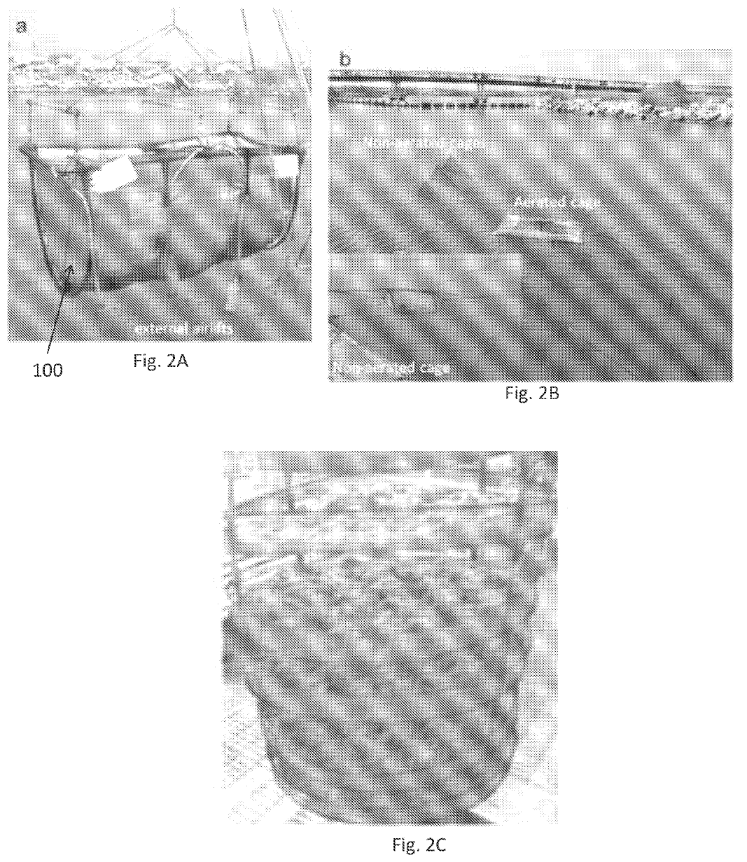

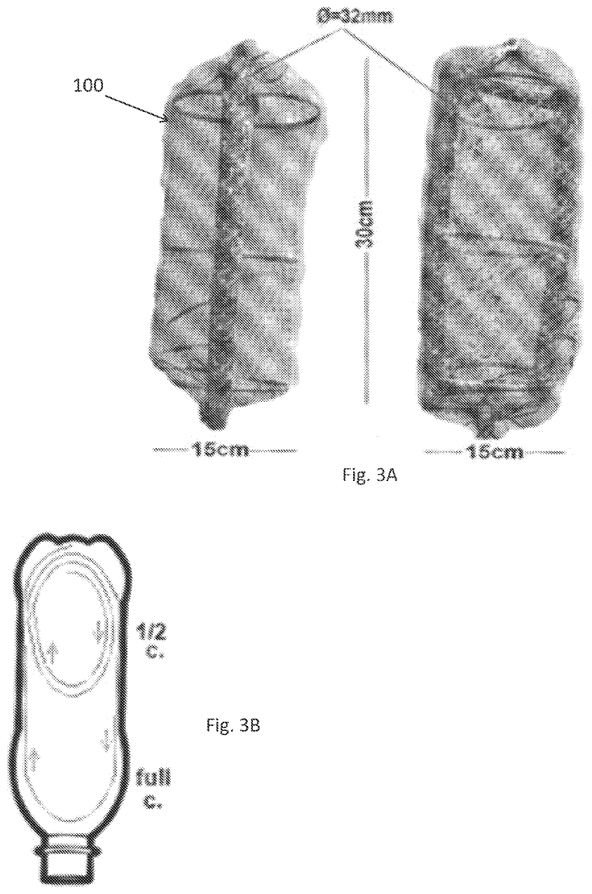

The system was installed ˜30 m from the shore (FIGS. 2A-2B). The average streamflow at that point was measured and found to be in the range of 6-8 cm s⁻¹. Air was supplied from 6 am to 6 pm through a central bottom pipe through 2 mm holes. For harvesting, the reactor was removed from the water using pulleys and hanged up to remove excess water by gravitation, followed by air and solar drying of the biomass (FIG. 2C).

Two experiments were performed. The first experiment started on Apr. 20, 2017 and ended on May 29, 2017; and the second experiment started on Jun. 15, 2017 and ended on Jul. 12, 2017. Sampling of a 2 kg biomass of algae was done every two weeks. Daily growth rate (DGR %) was calculated according to the following equation:

$$D G R = \frac{1}{N} \cdot \frac{{m}_{o u t} - {m}_{i n}}{{m}_{i n}} \cdot 100 \%$$

where N is the number of days (d) between measurements, mₒᵤₜ is the dry weight (DW) measured in grams at the end of each growth period, and mᵢₙ is the DW (g) of the inoculum. A standard protocol was used for surface water removal by centrifuging the algal biomass in an electric centrifuge (Beswin Portable Washer Spin Dryer CE-88 (6.0 kg) 2800 RPM Stainless Steel Housing) until all surface water was removed (<1 mL separated). Drying was done at 40° C. till constant weight (<5% change in consequent measurements). Dry matter was determined by drying in 105° C. for 3 h.

### Extensive Cultivation

For extensive cultivation experiments, a 2 cm layer of thalli was placed between two layers of nets (TENAX Tubular nets for Mussel Breeding & Packaging Shellfish Polypropylene, mesh configuration—rhomboidal, 32 G 223 neutral. 74 N 140 green, Gallo Plastik, Italy) in the non-tumbled and un-mixed cultivation cage 100, which had free water exchange with the surrounding sea. The cage 100 (0.15×0.3 m, total illuminated area 0.045 m²) was built from polyethylene (D=32 mm) and high-density polyethylene (HDPE), (D=6 mm) pipes and a TENAX (Gallo Plastik, Italy) net (FIG. 3A) to allow for full illumination and prevent grazing of algae by fish. The cages 100 were connected to a rope and located ˜30 m from the shore, at a distance of ˜10 m from the aerated, tumbled and mixed cultivation cage. Differently from the aerated cage, the biomass was held at a depth of ˜10 cm in a single layer with no aeration supplied. Fresh weight (FW) of 20 g of Ulva was loaded to each cage every two weeks (after sampling).

Determining the Effect of Tumbling with Air on Ulva sp. Growth Rate at a Controlled On-Land Cultivation System

To better understand the effect of tumbling with air on the Ulva sp. biomass growth rate in controlled environment, the biomass was cultivated at polyethylene terephthalate (PET) plastic bottles (1.5 L) with modified caps, to allow for water exchange and air supply (FIG. 3B). Five grams of FW of Ulva were loaded per bottle. Artificial seawater (salinity 3.5%, pH 8.2) was supplied with 21.4 mg L⁻¹ of NH₄NO₃ and 4 mg L⁻¹ of H₃PO₄ (Haifa Chemicals, Israel). Air was supplied at 0.36 LPM per bottle from 6 am to 6 pm. Water with nutrients was changed daily. The total cultivation time was 7 days per experiment. Two separate experiments were conducted: The first was conducted from Jun. 12, 2018 to Jun. 19, 2018 (3 replicates for tumbled with air and mixing and 3 replicates for non-tumbled and un-mixed bottles). The second experiment was conducted from Jun. 19, 2018 to Jun. 26, 2018 (6 replicates for aerated and 6 replicates for non-aerated bottles). Thalli rotation velocity was measured for a single thallus for half and full cycle (FIG. 3B) in three bottles with a stopper watch.

### Solar Irradiance and Temperature

Solar irradiance and temperature were measured every 15 min, using Onset HOBO Pendant® Temperature/Light 64K Data Logger (Onset Inc, MA), installed at 40 cm depth inside the aerated cage with the biomass. An additional sensor was installed inside the flat, not aerated cage at ˜10 cm depth. For on-land system temperature and irradiance were measured in the water and outside of the bottles with 2 sensors. Lux conversion to μmoles m⁻² s⁻¹ was done by a multiplication of measured lux values by 0.019, a constant used for sun illumination (http://www.egc.com/useful_info_lighting.php).

### Nutrients Measurement at the Cultivation Site

To measure nutrients, 50 ml of water was sampled at the cultivation site every two weeks with biomass sampling/loading. Nutrient analysis was done less than one hour after sampling in duplicates Ammonia, nitrite, nitrate, and phosphate were quantified using SMARTS Colorimeter (LaMotte, MD) with kits and protocols supplied by the manufacturer (LaMotte, MD).

### Biomass Composition Analysis

For ash analysis, the biomass (DW) was ignited in a pre-weighed clean crucible at 550° C. for 3 h in a muffle furnace (Thermolyne muffle furnace, Thermo Scientific, MA). The crucibles were finally removed from the furnace, kept in a desiccator to cool them down at room temperature and weighed. The analysis was done in triplicate. Protein content was determined according to AOAC 981.10 with an automatic Kjeldahl system for total protein quantification. Protein calculation factor of 5 was used (Angell et al., 2016). The analysis was done by a certified food chemistry company (AminoLab, Rehovot Israel). For caloric value analysis, twenty gram (DW) of biomass, harvested on May 3 and 17, 2017, dried at 40° C. to constant weight, were analyzed for energy content according to ASTM D5865-13 (Standard Test Method for Gross Calorific Value of Coal and Coke) by a certified laboratory of Israel Electric company. Element analysis, CHNS was done using Thermo Scientific CHNS Analyzer (Flash2000) at The Technion, Israel Institute of technology chemical characterization and surface chemistry unit.

For monosaccharide quantification the biomass was hydrolyzed in 2% sulfuric acid, 1:20 solid to solvent ratio, 30 mM, 121° C., in 10 mL centrifuge tubes (Nalgene™ Oak Ridge High-Speed PPCO Centrifuge Tubes (Thermo-Fisher Scientific, CA)) in autoclave (Tuttnauer 2540MLV, Netherlands). Monosaccharide contents in the hydrolysates were quantified by HPAEC-PAD (High-Pressure Anion-Exchange Chromatography coupled with Pulsed Amperometric Detection) using a Dionex ICS-5000 platform (Dionex, Thermo Fischer Scientific, MA, USA) with an analytical column (Aminopack 10) and its corresponding guard column. An electrochemical detector with an AgCl reference electrode was used for detection. The analysis was performed using an isocratic flow of 4.8 mM KOH for 20 min. Then, the column was washed with 100 mM KOH between each run and re-equilibrated with 4.8 mM KOH prior to injection. The column temperature was kept at 30° C., and the flow rate was set to 0.25 mL min⁻¹. Calibration curves were produced for each sugar with internal standards. In this work, rhamnose, arabinose, galactose, glucose, xylose, glucuronic acid, mannitol, fucose, and mannose, were quantified. Glucuronic acid and uronic acid derivatives content were monitored by using a program that involved three eluents (NaOH, ultrapure water and sodium acetate) (Table 1). Two additional peaks were observed in all samples that were assumed to represent aldobiouronic acid and iduronic acid (reefed herein as uronic acid derivatives). Each algal sample was hydrolyzed in duplicate before analysis. Each of the hydrolysates was analyzed in duplicate on HPIC. All data is reported as the weight fraction of the specific monosaccharide biomass (μg of monosaccharide mg⁻¹ DW (Dry Weight) biomass).

TABLE 1
Eluent gradients of the HPIC program used for glucuronic acid monitoring

| | Eluent | | |
|---|---|---|---|
| | | | C % |
| | A % | B % | (sodium acetate 1M + |
| Time | (ultrapure water) | (NaOH 480 mM) | NaOH 100 mM) |
| 0 | 98 | 1 | 1 |
| 12 | 98 | 1 | 1 |
| 15 | 98 | 1 | 1 |
| 26 | 52 | 8 | 40 |
| 31 | 52 | 8 | 40 |
| 32 | 98 | 1 | 1 |
| 47 | 98 | 1 | 1 |

### Statistical Analysis

Statistical analysis was performed with Excel (ver. 13, Microsoft, WA) Data analysis package and R software (ver. 2015, RStudio Inc., Boston, Mass.). Standard deviation (±STDEV) is shown in error bars. For groups comparison, on-tail Student-t analysis was performed.

### Results and Discussion

### Environmental Parameters During Sea Cultivation

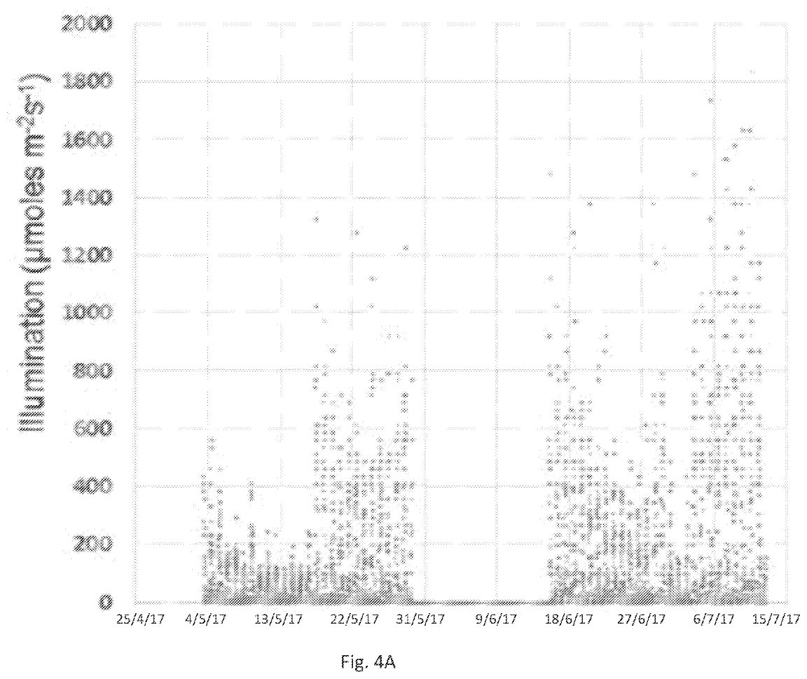

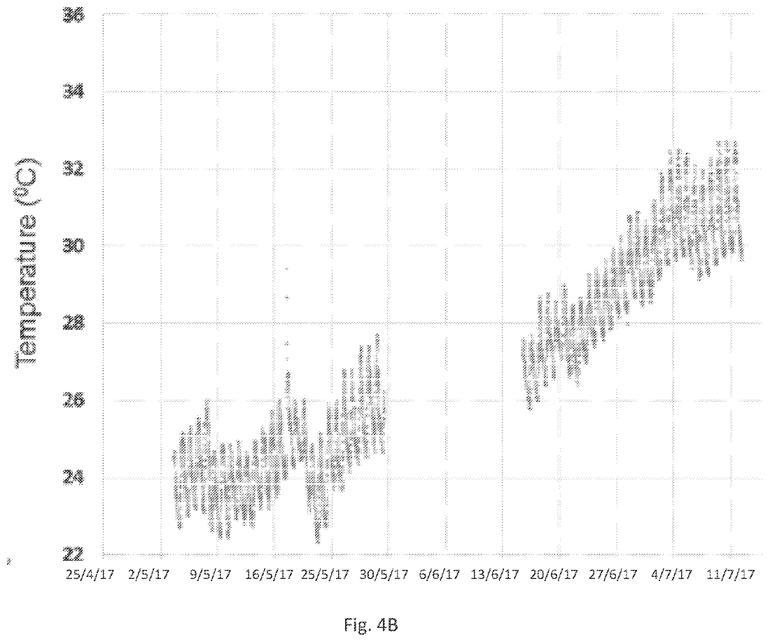

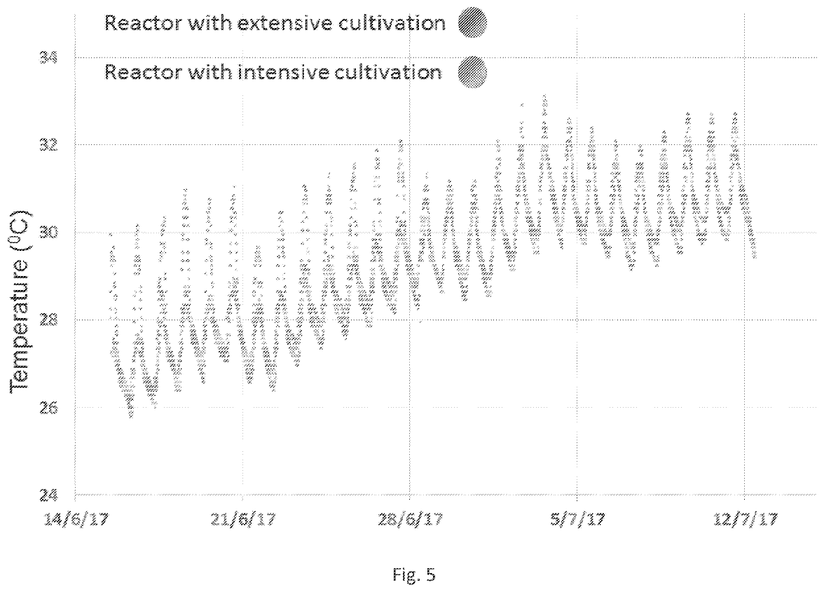

Illumination and temperature profile in an aerated reactor/cage at 40 cm depth is shown in FIGS. 4A and 4B. Importantly, the temperature in the cultivation cages increased from ˜24° C. to 32° C. during the cultivation period. Comparison between an average temperature in the mixed and un-mixed with aeration cages showed that until July an aerated cage was at least 2° C. cooler than a non-aerated cage (FIG. 5). This is important, as the temperature at these levels (close to 30° C.) slows Ulva sp. Growth (De Casablanca et al., 1998; De Casabianca et al., 2002). Measured nutrients levels are shown in Table 2 (data shown is an average of a duplicate measurement). Large fluctuations in nutrients levels (NH₃ 0.09-2.16 ppm, NO₃⁻ 0.44-2.11 ppm, NO₂ 0.13-1.53, and PO₄⁻³ 0.05-0.99 ppm) were observed.

TABLE 2
Nutrients levels measured at the cultivation site.

| Measured date | NH₃ (ppm) | NO₃⁻ (ppm) | NO₂ (ppm) | PO₄⁻³ (ppm) |
|---|---|---|---|---|
| 20 Apr. 2017 | 0.61 | 0.44 | 0.20 | 0.06 |
| 3 May 2017 | 0.64 | 0.84 | 0.07 | 0.10 |
| 9 May 2017 | 2.16 | 0.88 | 0.69 | 0.99 |
| 29 May 2017 | 0.04 | 0.57 | 0.66 | 0.11 |
| 15 Jun. 2017 | 0.62 | 1.32 | 0.13 | 0.05 |
| 28 Jun. 2017 | 0.09 | 2.11 | 0.69 | 0.09 |
| 12 Jul. 2017 | 0.94 | 1.36 | 1.52 | 0.08 |

### Growth Rates and Area Productivity

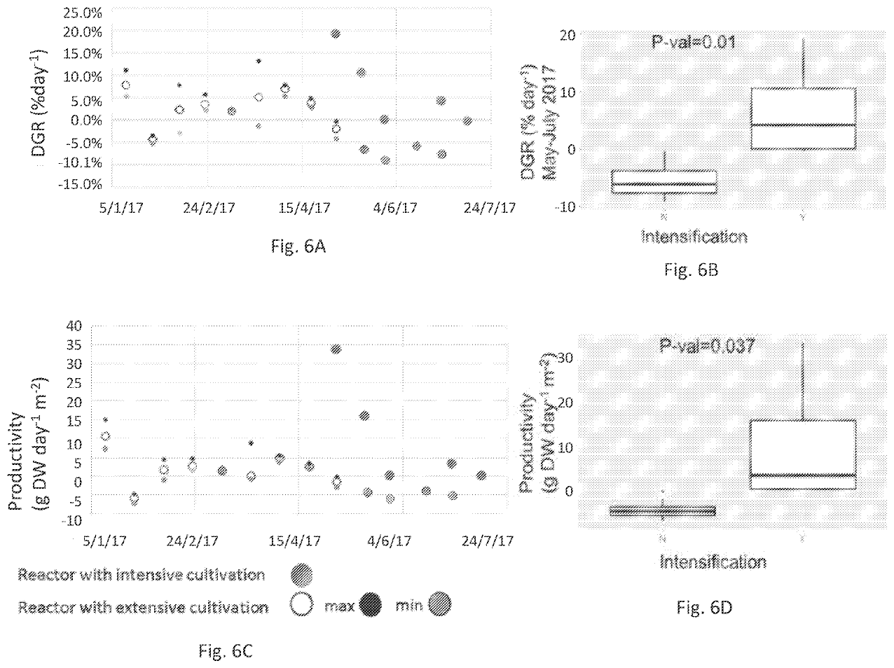

Biomass was weighed every two weeks and the yield was harvested. In the first and second experiments, the highest growth rates (19.2% and 4.1%) were measured after the first two weeks of cultivation. This could be the result of accumulated nutrients during inoculation for both groups. In addition, higher levels of NH₃ and NO₃⁻ were observed on May 9, 2017, Jun. 15, 2017, and Jun. 28, 2017 and could support the growth. Higher growth rates were observed at lower temperatures (10.6-19.2% DGR were observed when the temperatures were at 24-26° C.) and lower growth rates were observed at higher temperatures (26-32° C.). Interestingly, the growth rates were higher in the intensified cultivation cage compared to the extensive one at the same season (FIG. 6A). Consequently, tumbling of macroalgae with air, mixing and supplying external water to the cultivation cages (referred to herein as intensification cultivation) in the sea led to the highest yield observed in the entire season (FIG. 6C and Table 3 below). The highest yield was observed after 13 days of cultivation, on May 3, 2017, and it was 33.72 g DW day⁻¹ m⁻² (6.74 g C day⁻¹ m⁻², 0.33 g N day⁻¹ m⁻²), or 37.79 g DW day⁻¹ m⁻³. In comparison, the highest yield in the extensive cultivation from January 2017 to July 2017 was 15 g DW day⁻¹ m⁻² in January 2017. There was no growth from May to July in the cages with extensive cultivation (FIG. 6A), probably due to high temperature (FIG. 5).

Importantly, cultivation in the tumbled with air cultivation cages with mixing but without external water supply from outside the cage (FIG. 1C) resulted in biomass loss when growth was observed in the extensive system from January to May 2017. This implies that tumbling, air mixing, and external water flux, which provides nutrients and colder water, play a combined role in growth rate intensification. It is difficult to decompose to what extent each component contributes to the growth rate intensification due to complex interactions between the components, such as the reduction of photo-inhibition, enhanced supply of nutrients, enhanced gas exchange and hydrodynamic stimulus. Furthermore, air and biomass movement in the reactor might also prevent the development of damaging viruses or bacteria. In addition, water supply using airlift pumps 102 from deeper water layers will typically reduce the temperature in the cultivation reactor 100, working against the temperature inhibition factor.

TABLE 3
Ulva sp. biomass growth rates and yields in intensification cultivation cages (two
separate experiments were performed)

| | | | | | Growth Rate | |
|---|---|---|---|---|---|---|
| | Density (FW) | | | | DGR in | |
| | In the cage with | | Yield in the cage with | | intensification | DGR in |
| | intensification | | itensification | | cage | extensive |
| Date | g FW m⁻² | g FW m⁻³ | gDW day⁻¹ m⁻² | gDW day⁻¹ m⁻³ | % day⁻¹ | cages % day⁻¹ |
| Apr. 20, 2017 | 1,174* | 1,315* | | | | |
| May 3, 2017 | 4,096 | 4,590 | 33.72 | 37.79 | 19.2% | −4.2% |
| May 17, 2017 | 2,482 | 2,781 | 15.88 | 17.79 | 10.6% | −6.7% |
| May 29, 2017 | 1,000 | 1,120 | 0 | 0 | 0.0% | −9.1% |
| Jun. 15, 2017 | 1,165* | 1,306* | | | | |
| Jun. 28, 2017 | 1,786 | 2,001 | 3.11 | 3.47 | 4.1% | −5.9% |
| Jun. 12, 2017 | 954 | 1,069 | | | −0.3% | Not measured |

FW-fresh weight.
DW-dry weight.
*Stays for initial density, the rest is density at harvesting.

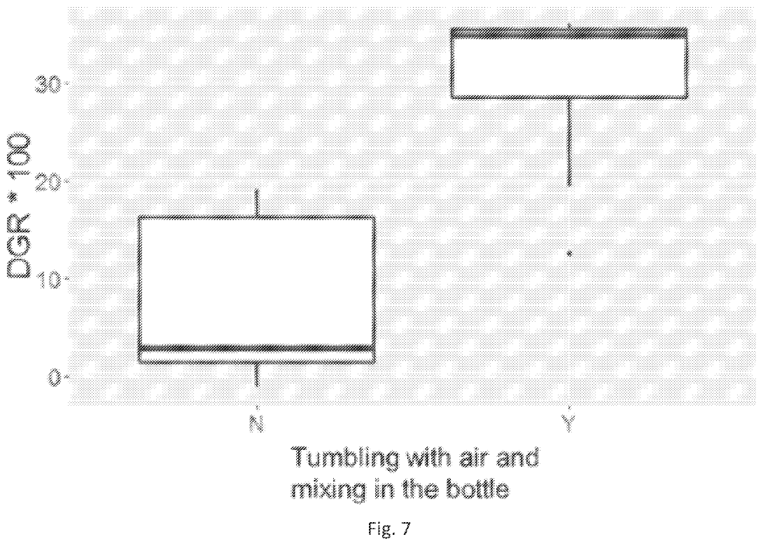

To shed light on some of the aforementioned coupled interactions, a series of experiments in on-land system were performed where the only changed parameters between cultivation reactors were aeration. The experiments in the controlled environment showed that under air temperature between 21.9° C. (at night) and 40.53° C. (during the day), water temperature 21.6° C. (at night) and 37.4° C. (during the day), and maximum solar illumination intensity (outside the reactors) of 942 μmoles m⁻² s⁻¹, mixing by aeration (5.8-8.6 rpm) increased the DGR from 7.6±2.6% to 29.9±2.9% (p-val. <2.8·10⁻⁵). These results in the controlled land-based system showed that tumbling with air and mixing increase the growth rates of Ulva sp. biomass (FIG. 7) when nutrients are available in excess, corroborating that a combination of tumbling with air, mixing and nutrients supply are needed for the intensification of growth, when other parameters, such as illumination and temperature are equal.

Previous studies on Ulva sp. cultivation offshore reported on the maximum 17% DGR when the algae were cultivated downstream from fish cages and −15% upstream the cages (Korzen et al., 2015). In addition, previous works also compared various methods for Ulva sp. growth in on-land tank, ropes and spray systems. Studies with Ulva sp. growth in tanks with tumbling reported on DGR of 10-45% (Msuya et al., 2010; Bruhn et al., 2011; Angell et al., 2014; Gomez Pinchetti et al., 1998; Copertino et al., 2009; Hiraoka et al., 2008), with high values achieved in nitrogen-rich wastewaters such as manure streams (Nielsen et al., 2012).

Compositional Analysis of Cultivated Ulva sp. Biomass

Ash content of the dry matter of samples with the highest yields 33.72 (g DW day⁻¹ m⁻²) and 15.86 (g DW day⁻¹ m⁻²), harvested on May 3 and 17, 2017 from cages with intensified cultivation, was 38.47±0.01% and 37.87±0.01%, respectively.

The harvested biomass had significantly lower protein content in comparison with biomass grown in laboratory conditions (2.9-6.2% protein in the intensified cages in the sea, 0.53-9.08% in cages with extensive cultivation v. 33% in the lab). Low protein (5.9-17%) has been reported for multiple natural stocks of various Ulva species, suggesting that precise nitrogen control is required to maintain high protein content.

The elementary composition of the harvested biomass from cages with intensified cultivation varied as follows during the entire cultivation period: C % 19.6-22.5; H % 3.7-4.6, N % 0.65-1.4 and S % 3.54-6.74 (Table 4) and were not significantly different from the composition of the biomass cultivated in extensive cages (Table 5). These results indicate the potential of Ulva sp. to capture carbon and nitrogen, two important climate change factors, using offshore cultivation at 6.74 gC m⁻² d⁻¹ (25 g CO₂ m⁻² d⁻¹) and 0.34 gN m⁻² d⁻¹, at maximum growth rate achieved in this study (assuming 0.15 DW: FW ratio). According to the Israel Ministry of Environmental Protection, the national Israel GHG emission reduction target for 2030 is 2.7 ton CO₂ per capita (26% from the emissions in 2005). Therefore, ˜0.108 km² of the sea area should be allocated per capita, if macroalgae Ulva sp. with intensified cultivation are used to achieve these goals.

TABLE 4
Protein content and elementary composition of harvested
Ulva biomass from a tumbled with air cultivation cage.

| Harvesting | Cultivation | Protein | C | H | N | S |
|---|---|---|---|---|---|---|
| date | days | content (%) | (%) | (%) | (%) | (%) |
| May 3, 2017 | 13 | 5.28 | 19.9 | 4.6 | 1.05 | 6.74 |
| May 17, 2017 | 27 | 2.96 | 21.3 | 4.4 | 0.65 | 6.79 |
| May 29, 2017 | 39 | 4.64 | 21.3 | 4.1 | 0.98 | 3.54 |
| Jun. 28, 2017 | 13 | 4.8 | 22.5 | 4.6 | 1.06 | 5.52 |
| Jul. 12, 2017 | 27 | 6.24 | 19.6 | 3.7 | 1.40 | 3.15 |

* Protein was measured according to AOAC 981.10 with a multiplication factor of 5.
** CHNS shows the average of at least two technical repeats.

Analysis of carbohydrate content (Tables 6-7) showed that glucose was the major carbohydrate, followed by rhamnose, xylose and uronic acid derivatives, which is consistent with our previous data on the same species harvested from the sea and cultivated in an on-shore reactor integrated into the building. Longer offshore cultivation in intensification cultivation cages (39 days v. 18 days) lead to lower content of fructose, glucose, rhamnose, uronic acid derivatives and xylose (Table 5). As the total carbon content of the biomass did not change with cultivation time (Table 4), we suggest that under offshore cultivation, the carbon is stored in fibers such as cellulose, which is not hydrolyzed by the presently used protocol.

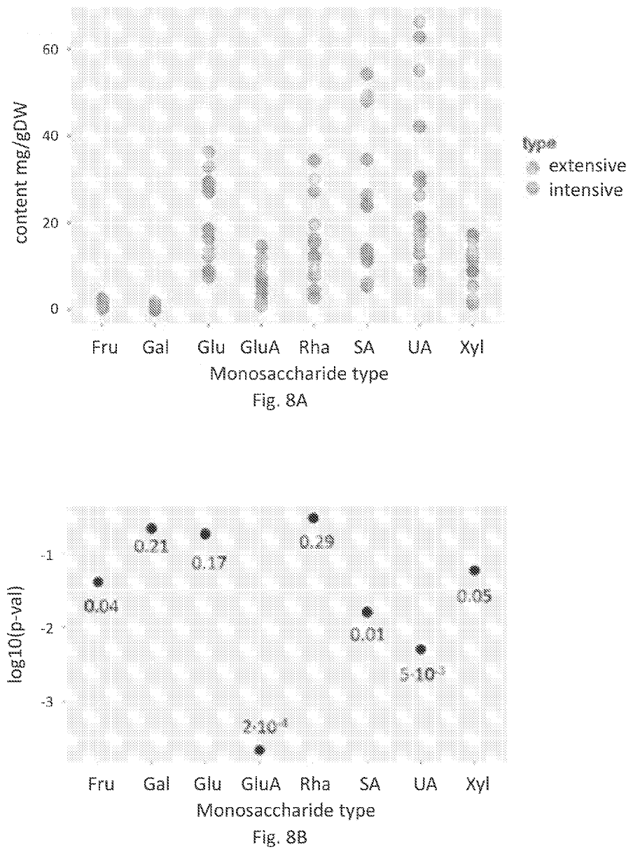

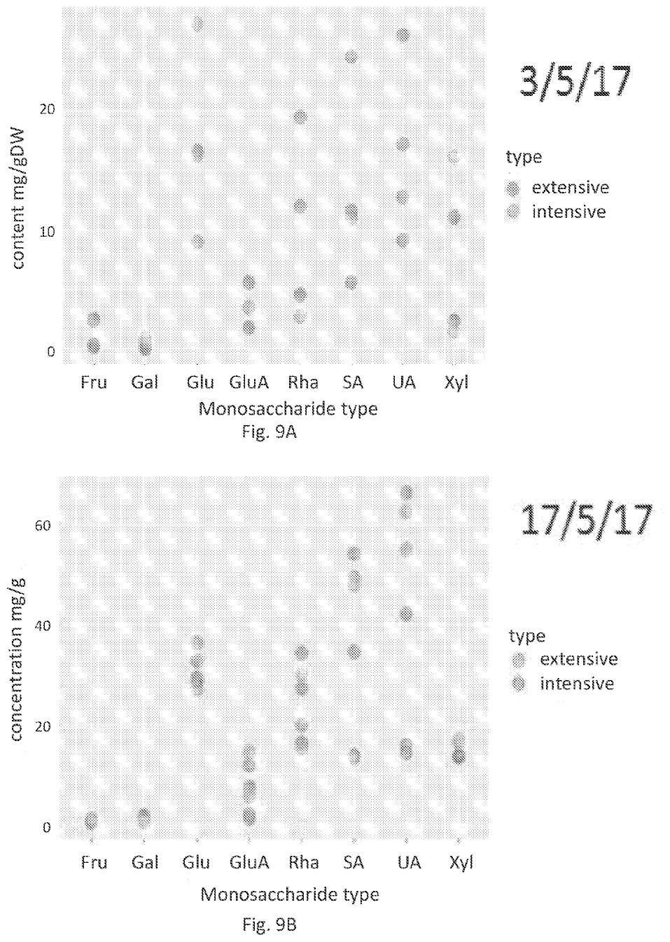

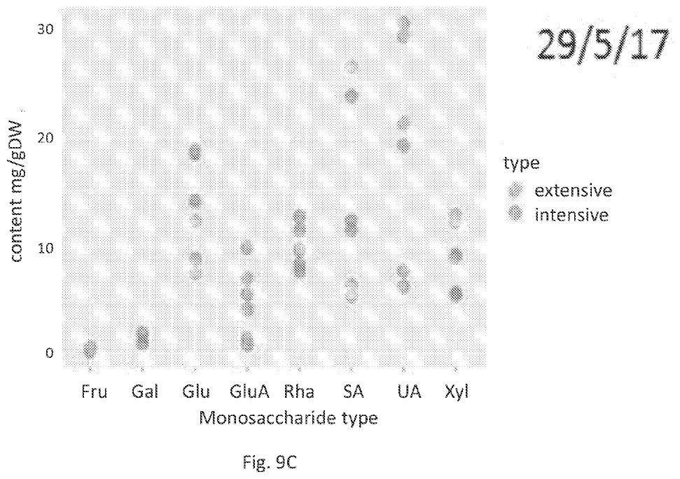

Comparison of the monosaccharide content of the matched biomass harvested on May 3, 17 and 29, 2017 (FIGS. 9A-9C, Tables 6-7) shows that intensification of cultivation led to a significant (p-val<0.5) increase in fructose content, and significant decrease ((p-val. <0.5) in glucuronic, uronic and other sugar acids (FIGS. 8A-8B).

TABLE 5
Protein content and elementary composition of harvested
Ulva biomass from an extensive cultivation cage

| | | Protein | | | | |
|---|---|---|---|---|---|---|
| Harvesting | Cultivation | content | C | H | N | S |
| date | days | (%) | (%) | (%) | (%) | (%) |
| Jan. 12, 2017 | 14 | 8.81 | 17.59 | 3.94 | 1.76 | 4.79 |
| Jan. 26, 2017 | 14 | 9.08 | 22.99 | 4.13 | 1.82 | 1.73 |
| Feb. 13, 2017 | 17 | 8.55 | 17.65 | 3.84 | 1.71 | 4.81 |
| Feb. 28, 2017 | 15 | 8.60 | 18.92 | 3.68 | 1.72 | 4.04 |
| Mar. 23, 2017 | 14 | 7.07 | 21.81 | 4.21 | 1.41 | 4.93 |
| Apr. 6, 2017 | 14 | 5.06 | 15.59 | 3.22 | 1.01 | 3.53 |
| Apr. 20, 2017 | 16 | 3.83 | 20.81 | 4.24 | 0.77 | 4.71 |
| May 3, 2017 | 13 | 0.53 | 5.73 | 2.27 | 0.11 | 3.18 |
| May 17, 2017 | 14 | 2.62 | 22.81 | 4.41 | 0.52 | 5.84 |
| May 18, 2017 | 14 | 4.32 | 24.16 | 4.44 | 0.86 | 3.30 |
| May 26, 2017 | 10 | 6.07 | 19.59 | 4.09 | 1.21 | 5.28 |
| May 29, 2017 | 10 | 3.88 | 22.40 | 4.08 | 0.78 | 3.64 |
| Jul. 17, 2017 | 14 | 4.71 | 21.89 | 3.93 | 0.94 | 3.55 |

* Protein was measured according to AOAC 981.10 with a multiplication factor of 5.
** CHNS shows the average of at least two technical repeats

TABLE 6
Monosaccharides content of Ulva sp. biomass cultivated with intensification
achieved with tumbling with air, mixing and external water supply

| | | | | | | Uronic acid | |
|---|---|---|---|---|---|---|---|
| Harvesting | Cultivation | Fructose | Galactose | Glucose | Rhamnose | derivatives | Xylose |
| date | days | mg/g DW | mg/g DW | mg/g DW | mg/g DW | mg/g DW | mg/g DW |
| May 3, 2017 | 13 | 2.49 ± 0.15 | 0.76 ± 0.25 | 21.76 ± 7.84 | 15.70 ± 5.32 | 19.58 ± 9.57 | 13.64 ± 3.55 |
| May 17, 2017 | 27 | 0.96 ± 0.13 | 1.02 ± 0.55 | 29.42 ± 0.08 | 15.85 ± 0.74 | 15.23 ± 0.44 | 13.71 ± 0.34 |
| May 29, 2017 | 39 | 0.52 ± 0.03 | 1.80 ± 0.01 | 18.58 ± 0.17 | 9.61 ± 0.17 | 6.93 ± 0.70 | 12.47 ± 0.44 |
| Jun. 28, 2017 | 13 | 3.33 ± 2.65 | 2.26 ± 0.70 | 39.16 ± 2.57 | 21.91 ± 2.57 | 18.05 ± 0.67 | 16.72 ± 2.31 |
| Jul. 12, 2017 | 27 | 1.95 ± 0.11 | 3.80 ± 0.85 | 18.43 ± 2.59 | 12.47 ± 2.59 | 5.59 ± 0.04 | 9.08 ± 1.56 |

TABLE 7
Monosaccharides content of Ulva sp. biomass cultivated under extensive
cultivation

| | | | | | | Uronic acid | |
|---|---|---|---|---|---|---|---|
| | Cultivation | Fructose | Galactose | Glucose | Rhamnose | derivatives | Xylose |
| | days | mg/g DW | mg/g DW | mg/g DW | mg/g DW | mg/g DW | mg/g DW |
| Jan. 12, 2017 | 14 | 0.35 ± 0.29 | 1.6 ± 0.42 | 6.67 ± 8.22 | 16.43 ± 4.15 | 67.43 ± 25.64 | 7.68 ± 1.89 |
| Jan. 26, 2017 | 14 | 0.35 ± 0.04 | 0.31 ± 0.03 | 15.58 ± 1.26 | 7.1 ± 0.54 | 21.33 ± 3.84 | 4.97 ± 0.18 |
| Feb. 13, 2017 | 17 | 0.44 ± 0.09 | 0.5 ± 0.4 | 7.73 ± 4.64 | 6 ± 3.76 | 29.24 ± 13.65 | 3.4 ± 1.58 |
| feb. 28, 2017 | 15 | 0.36 ± 0.07 | 0.24 ± 0.51 | 5.9 ± 6.2 | 11.39 ± 2.87 | 39.72 ± 1.42 | 4.96 ± 0.06 |
| Mar. 23, 2017 | 14 | 1.01 ± 0.95 | 0.94 ± 0.01 | 23.79 ± 1.17 | 13.13 ± 0.03 | 49.77 ± 2.63 | 6.37 ± 0.02 |
| Apr. 6, 2017 | 14 | 0.46 ± 0.03 | 0.3 ± 0.19 | 9.39 ± 0.93 | 5.42 ± 0.5 | 16.01 ± 0.09 | 3.77 ± 0.17 |
| Apr. 20, 2017 | 16 | 0.58 ± 0.2 | 1.16 ± 0.04 | 48.02 ± 2.85 | 23.58 ± 0.88 | 71.44 ± 0.68 | 10.85 ± 0.11 |
| May 3, 2017 | 13 | 0.3 ± 0.1 | 0.09 ± 0.04 | 12.84 ± 5.41 | 3.68 ± 1.22 | 13.19 ± 5.67 | 1.93 ± 0.63 |
| May 17, 2017 | 14 | 1.05 ± 0.17 | 1.12 ± 0.23 | 32.27 ± 5.53 | 23.59 ± 5.22 | 48.52 ± 9.15 | 15.61 ± 2.32 |
| May 18, 2017 | 14 | 0.55 ± 0.04 | 1.66 ± 0.22 | 29.91 ± 4.12 | 32.2 ± 3.05 | 64.39 ± 2.59 | 16.92 ± 0.52 |
| May 26, 2017 | 10 | 0.23 ± 0.03 | 0.91 ± 0.01 | 8.07 ± 0.91 | 12.03 ± 0.91 | 29.88 ± 0.86 | 5.49 ± 0.09 |
| May 29, 2017 | 10 | 0.34 ± 0.03 | 1.01 ± 0.02 | 13.12 ± 1.33 | 7.93 ± 0.36 | 20.22 ± 1.43 | 8.91 ± 0.2 |

Energy Content of Ulva sp. Biomass Cultivated with Intensification

The energetic high heating value (HHV) of dried biomass as fuel was 8.46 MJ kg_{DW}⁻¹ (remained moisture (RM %) 11.21% for the harvest done on May 3 and 9; 13 MJ kg_{DW}⁻¹ (remained moisture (RM %) 13.79% for the harvest done on May 17. Hence, at the observed maximum, Ulva sp. biomass can produce 2 MJ m⁻² per day or produce a maximum power density of 23 W m⁻² for direct combustion.

### Conclusions

The present work tested the feasibility of offshore cultivation of Ulva sp. biomass in an intensified offshore reactor, with tumbling and mixing with air and external water supply. It was shown that this intensification method allowed the production of Ulva sp. biomass during May-July in 2017 while no growth was seen in extensive offshore systems. In addition, tumbling with air and mixing increased the growth rate of Ulva sp. in the controlled, land-based systems in comparison with the same system without tumbling and mixing. Multiple coupled mechanisms can lead to these changes, including the reduction of photo-inhibition, enhances nutrient flux, the water, enhanced gas exchange, and hydrodynamic stimulus. Air and biomass movement in the reactor might also prevent the development of axenic zones, and the development of detrimental levels of viruses or bacteria or excretion of growth inhibiting photosynthesis by-products. In addition, water supply using airlift pumps from deeper layers reduces the temperature of the reactor. Our findings open new directions for the design of offshore cultivation systems that will produce usable biomass without arable land and fresh water.

### REFERENCES

Angell A R, Mata L, de Nys R, Paul N A. The protein content of seaweeds: a universal nitrogen-to-protein conversion factor of five. J Appl Phycol [Internet]. 2016 February; 28(1):511-24
Angell A R, Mata L, de Nys R, Paul N A. Variation in amino acid content and its relationship to nitrogen content and growth rate in Ulva ohnoi (Chlorophyta). J Phycol. 2014; 50(1):216-26
Ben Yahmed N, Jmel M A, Ben Alaya M, Bouallagui H, Marzouki M N, Smaali I. A biorefinery concept using the green macroalgae Chaetomorpha linum for the coproduction of bioethanol and biogas. Energy Conyers Manag. 2016 July; 119:257-65
Bird K. Cost analyses of energy from marine biomass. In: Bi. In: Seaweed Cultivation for Renewable Resources. 1987a. p. 327-50
Bird K. Tropical macroalgal cultivation for bioconversion to methane Energy from Biomass and Wastes X. Institute of Gas Technology, Chicago, In p. 1283-92 1987b
Buck B H, Buchholz C M. The offshore-ring: A new system design for the open ocean aquaculture of macroalgae. J Appl Phycol [Internet]. 2004; 16(5):355-68
Buck B H, Langan R. Aquaculture Perspective of Multi-Use Sites in the Open Ocean [Internet]. Buck B H, Langan R, editors. Cham: Springer International Publishing; 2017
Bruhn A, Dahl J, Nielsen H B, Nikolaisen L, Rasmussen M B, Markager S, et al. Bioenergy potential of Ulva lactuca: biomass yield, methane production and combustion. Bioresour Technol [Internet]. 2011; 102(3):2595-604
Camus C, Infante J, Buschmann A H. Overview of 3 year precommercial seafarming of Macrocystis pyrifera along the Chilean coast. Reviews in Aquaculture. 2016
Copertino M D S, Tormena T, Seeliger U. Biofiltering efficiency, uptake and assimilation rates of Ulva clathrata (Roth) J. Agardh (Clorophyceae) cultivated in shrimp aquaculture waste water. J Appl Phycol. 2009; 21(1):31-45
Cuaresma M, Janssen M, van den End E J, Vílchez C, Wijffels R H. Luminostat operation: A tool to maximize microalgae photosynthetic efficiency in photobioreactors during the daily light cycle? Bioresour Technol. 2011; 102(17):7871-8
De Casabianca M L, Barthelemy N, Serrano O, Sfriso A. Growth rate of Ulva rigida in different Mediterranean eutrophicated sites. Bioresour Technol. 2002; 82(1):27-31.
De Casablanca M L, Posada F. Effect of Environmental Parameters on the Growth of Ulva rigida (Thau Lagoon, France). Bot Mar. 1998; 41:157-65
Fernand F, Israel A, Skjermo J, Wichard T, Timmermans K R, Golberg A. Offshore macroalgae biomass for bioenergy production: Environmental aspects, technological achievements and challenges. Renew Sustain Energy Rev. 2016
Golberg A, Liberzon A. Modeling of smart mixing regimes to improve marine biorefinery productivity and energy efficiency. Algal Res [Internet]. 2015; 11:28-32
Golberg A, Linshiz G, M K, Hillson N J, Chemodanov A, Koudritsky M, et al. Distributed marine biorefineries for developing economies. IMECE2012-86051 Proceeding ASME Congr Exhib [Internet]. 2012
Golberg A, Vitkin E, Linshiz G, Khan S A, Hillson N J, Yakhini Z, et al. Proposed design of distributed macroalgal biorefineries: Thermodynamics, bioconversion technology, and sustainability implications for developing economies. Biofuels, Bioprod Biorefining [Internet]. 2014; 8:67-82
Gómez Pinchetti J L, del Campo Fernandez E, Moreno Díez P, Reina G G. Nitrogen availability influences the biochemical composition and photosynthesis of tank-cultivated Ulva rigida (Chlorophyta). J Appl Phycol [Internet]. 1998; 10(4):383-9
Hiraoka M, Oka N. Tank cultivation of Ulva prolifera in deep seawater using a new “germling cluster” method. J Appl Phycol. 2008; 20(1):97-102
Hirayama S, Miyasaka M, Amano H, Kumagai Y, Shimojo N, Yanagita T, et al. Functional sulfur amino add production and seawater remediation system by sterile Ulva sp. (Chlorophyta). Appl Biochem Biotechnol—Part A Enzym Eng Biotechnol. 2004
Jiang L, Luo S, Fan X, Yang Z, Guo R. Biomass and lipid production of marine microalgae using municipal wastewater and high concentration of CO₂. Appl Energy. 2011; 88(10):3336-41 Jung K A a., Lim S-R R, Kim Y, Park J M M. Potentials of macroalgae as feedstocks for biorefinery. Bioresour Technol. 2013; 135:182-90
Korzen L, Abelson A, Israel A. Growth, protein and carbohydrate contents in Ulva rigida and Gracilaria bursa-pastoris integrated with an offshore fish farm. J Appl Phycol [Internet]. 2015
Krupnik, N, Paz, G, Douek, J, Lewinsohn, E, Israel, A, Mineur, F, Maggs C. Native and invasive Ulva species from the Israeli Mediterranean Sea: risk and potential. Mediterr Mar Sci PressGoh C S, Lee K T. A visionary and conceptual macroalgae-based third-generation bioethanol (TGB) biorefinery in Sabah, Malaysia as an underlay for renewable and sustainable development. Renew Sustain Energy Rev. 2010; 14(2):842-8
Lehahn Y, Ingle K N, Golberg A. Global potential of offshore and shallow waters macroalgal biorefineries to provide for food, chemicals and energy: feasibility and sustainability. Algal Res. 2016; 17:150-60
Msuya F E, Neori A. The Performance Of Spray-Irrigated Ulva Lactuca (Ulvophyceae, Chlorophyta) As A Crop And As A Biofilter Of Fishpond Effluents. J Phycol [Internet]. 2010; 46(4):813-7
Nielsen M M, Bruhn A, Rasmussen M B, Olesen B, Larsen M M, Møller H B. Cultivation of Ulva lactuca with manure for simultaneous bioremediation and biomass production. J Appl Phycol. 2012; 24(3):449-58
Roesijadi A G, Copping A, Huesemann M. Techno-Economic Feasibility Analysis of Offshore Seaweed Farming for Bioenergy and Biobased Products. 2008
Skjermo J, Aasen I, Arff J, Broch O, Carvajal A. A new Norwegian bioeconomy based on cultivation and processing of seaweeds: Opportunities and R&D needs. 2014
Suganya T, Varman M, Masjuki H H, Renganathan S. Macroalgae and microalgae as a potential source for commercial applications along with biofuels production: A biorefinery approach. Vol. 55, Renewable and Sustainable Energy Reviews. 2016. p. 909-41
Zijffers J W F, Schippers K J, Zheng K, Janssen M, Tramper J, Wijffels R H. Maximum Photosynthetic Yield of Green Microalgae in Photobioreactors. Mar Biotechnol. 2010; 12(6):708-18

1. An apparatus for growing/cultivating macroalgae in a body-of-water, preferably in the sea/offshore, the apparatus comprising:
a) a growing/cultivation cage/reactor for positioning in the body-of-water, having permeable walls and bottom enabling free flow of water, gas and nutrients from the body-of-water into the growing cage and vise-versa; and
b) a macroalgae suspending and mixing system designed to mix/tumble/suspend water in the cage from bottom to top and consequently the macroalgae grown therein, by streaming gas from bottom of the cage via gas flow outlets,
wherein the apparatus is designed for free-floating growing of said macroalgae.

2. The apparatus of claim 1, further comprising: an artificial light source and/or a water-pump for water exchange in the growing cage.

3. (canceled)

4. The apparatus of claim 1, further comprising at least one external airlift for water exchange and optionally turbulence enhancement in the growing cage.

5. (canceled)

6. The apparatus of claim 1, further comprising a heating/cooling unit associated with a thermostat measuring the water temperature within the growing cage.

7. The apparatus of claim 1, wherein said permeable walls are non-selective permeable walls.

8. The apparatus of claim 1, wherein said permeable walls further enable penetration of light into the cage.

9. The apparatus of claim 1, wherein said growing cage is a closed cage further having a non-selective permeable and transparent cover.

10. The apparatus of claim 1, wherein said permeable walls and bottom are made of a mesh designed to prevent macroalgae from exiting the cage and fish from entering the cage and/or grazing on the macroalgae through the mesh.

11. The apparatus of claim 1, wherein said suspending and mixing system comprises a gas-blowing mechanism and gas pipes positioned essentially at the bottom of the cage.

12. The apparatus of claim 1, further comprising a floatation device/mechanism for maintaining said cage floating at water-surface, or at a desired depth at which the upper surface of the water in the cage is still exposed to sunlight.

13. The apparatus of claim 12, further comprising a light sensor designed to measure the amount of light within the cage and: (i) adjust the floating level of the cage accordingly using said floatation device/mechanism; and/or (ii) activate an artificial light source when present.

14. The apparatus of claim 1, further associated with a computing system comprising a memory and a processor designed to control any one of: said macroalgae suspending and mixing system; said floatation mechanism when present; said artificial light source when present; said water-pump when present; said at least one external airlift when present; and/or said heating/cooling unit when present.

15. An apparatus for growing macroalgae in a body-of-water, preferably in the sea/offshore, the apparatus comprising:
a) a growing/cultivation cage/reactor for positioning in the body-of-water, having permeable walls and bottom enabling free flow of water, gas and nutrients from the body-of-water into the growing cage and vise-versa;
b) a macroalgae suspending and mixing system designed to mix/tumble/suspend water in the cage from bottom to top and consequently the macroalgae grown therein, by streaming gas from bottom of the cage via gas flow outlets;
c) a floatation device/mechanism for maintaining said cage floating at water-surface, or at a desired depth at which the upper surface of the water in the cage is still exposed to sunlight;
d) at least one external airlift for water exchange and optionally turbulence enhancement in the growing cage; and
e) a water-pump for water exchange in the growing cage,
wherein the apparatus is designed for free-floating growing of said macroalgae.

16. The apparatus of claim 15, further comprising at least one of:
an artificial light source and optionally a light sensor designed to measure the amount of light within the cage and: (i) adjust the floating level of the cage accordingly using said floatation device/mechanism; and/or (ii) activate said artificial light source;
a power source;
a heating/cooling unit associated with a thermostat measuring the water temperature within the growing cage; and
a computing system comprising a memory and a processor designed to control any one of: said floatation mechanism; said artificial light source when present; said water-pump; said at least one external airlift; and/or said heating/cooling unit when present.

17. A method for growing/cultivating macroalgae in a body-of-water, preferably in the sea/offshore, the method comprising the steps of:
(i) positioning in the body-of-water an apparatus according to claim 1;
(ii) placing an inoculum of macroalgae in the growing/cultivation cage/reservoir cage; and
(iii) activating the macroalgae suspending and mixing system for tumbling/suspending the water in the cultivation cage from bottom to top to thereby expose in a cyclic manner different portions of the water in the cage, and consequently the macroalgae grown therein, to sunlight,
wherein: (1) the amount, intensity and speed of gas streamed into the cultivation cage by said suspending and mixing system is determined according to the type, density, and growing stage of said macroalgae; and (2) said tumbling is conducted continuously until a desired density/amount of the macroalgae in the cultivation cage is achieved.

18. The method of claim 17, further comprising a final step of harvesting the macroalgae.

19. The method of claim 17, wherein said apparatus comprises said water-pump and/or said at least one external airlift, and said method further comprises a step of activating said water-pump and/or said at least one external airlift for water exchange enhancement in the cultivation cage, and consequently enriching nutrient level therein.

20. The method of claim 17, further comprising a step of actively exchanging the water in the cultivation cage by pumping water into the cage, optionally from a remote location in the body-of-water.

21. The method of claim 17, further comprising a step of: (i) adjusting the water temperature within the cultivation cage; and/or adjusting the amount of light reaching the cultivation cage.

22. The method of claim 17, wherein step (i) of positioning an apparatus in the body-of-water means positioning the apparatus offshore.
